(19) United States
(12) Patent Application Publication
(10) Pub. No.: US 2010/0226893 A1
(43) Pub. Date: Sep. 9, 2010

(54) High Retention Cell Injectate

(75) Inventor: Julie Trudel, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/400,111

(22) Filed: Mar. 9, 2009

(51) Int. Cl. A61K 35/34; A61P 9/00

(52) U.S. Cl. 424/93.7

(57) ABSTRACT

The present disclosure generally relates to high retention cell injectates, methods of producing the high retention cell injectates and further to methods of treating diseases or conditions by administering the cell injectates to patients in need thereof. To allow for cell rentention into a provided hyaluronic acid solution, the present disclosure provides pretreatment of cells with one or more substances that increases the number of CD44 receptors on their surfaces. Cells expressing CD44 ligands show increase in binding to hyaluronic acid. The pretreated cells are thus incorporated into hyaluronic acid.

### FIELD OF THE INVENTION

The present disclosure relates to high retention cell injectates and associated methods for making the same and treating various diseases and conditions.

### BACKGROUND OF THE INVENTION

Throughout the course of modern medical history therapeutic agents have been administered to suffering patients to heal them or to at least mitigate the symptoms associated with various diseases and conditions. As one of ordinary skill in the art will appreciate, most therapeutic agents must be in contact with a target object to be effective.

For example, when penicillin and its antibacterial activity were discovered it was formulated to be taken into the body to eliminate harmful bacteria. Once taken into the body such as by oral intake, the penicillin molecules would be moved around by the circulatory system to come in contact with harmful bacteria. Once in contact, the β-lactam moiety (functional group) of penicillin binds to the enzyme (DD-transpeptidase) that links the peptidoglycan molecules in bacteria, which weakens the cell wall of the bacterium (in other words, the antibiotic causes cytolysis or death due to osmotic pressure). In addition, the build-up of peptidoglycan precursors triggers the activation of bacterial cell wall hydrolases and autolysins, which further digest the bacteria's existing peptidoglycan. The circulating blood in a suffering subject contains the introduced penicillin molecule and brings it to the target of interest which in this case is harmful bacteria so that it can carry out its function as a therapeutic agent.

Many orally ingested therapeutic agents and drugs are delivered to the target object by the circulating blood in a systemic fashion. Those drugs that cannot survive the hostile acidic environment of the stomach are often delivered intravenously but once in the circulatory system, the agents of cure or at least some relief, are delivered much the same way as orally ingested penicillin is delivered to harmful bacteria.

Under certain circumstances however, more targeted delivery of therapeutic agents is required than systemic dispersal. An example is cancer treatment. When therapeutic agents are cytotoxic and target cells to be destroyed are difficult to distinguish from a patient's normal cells, a more specific targeted delivery could mean the difference between a successful and failed treatment. When cytotoxic therapeutic agents such as paclitaxel can be specifically delivered to cancer cells without being in contact with normal cells the patient suffers fewer side effects such as hair loss.

Less targeted, systemic delivery may send a therapeutic agent to an unintended part of the body and actually cause harm. This is possible because of the body's circulatory system. The phenomenon of the carrying away from a therapeutic agent from the target site by circulation before desired action on the target site is known as washout. Washout often is a major problem to be overcome when considering targeted treatment of diseases or conditions such as ailments associated with the heart.

Recently, delivery of cells such as stem cells to target diseased areas has developed as an option for therapeutic treatment. This is because stem cells have the ability to reinvigorate themselves through mitotic cell division and can differentiate into a diverse range of specialized cell types. Currently, many medical researchers believe stem cell therapy has the potential to dramatically change the treatment of human disease. A number of adult stem cell therapies already exist, particularly bone marrow transplants that are used to treat leukemia.

Specific targeted delivery of cells such as stem cells to the treatment area can be a challenge. Circulating blood can wash out the stem cells that are injected to a target site such as a heart muscle wall. As a result, there is a significant need in the art for systems and methods that will enhance targeted delivery of therapeutic agents such as stem cells that overcomes the problem of washout and provide high retention.

### SUMMARY OF THE INVENTION

One of the most important challenges in the field of cell delivery is to permit a cell injectate to remain at the location where they have been delivered. Another important aspect of this problem consists in using a biocompatible carrier for cell delivery. Hyaluronic acid (hyaluronic acid, HA) solutions are known to be highly biocompatible and degrade over time. These solutions can thus be used as cell carriers. To allow for cell rentention into a provided hyaluronic acid solution, the present disclosure provides pretreatment of cells with one or more substances that increases the number of CD44 receptors on their surfaces. Cells expressing CD44 ligands show increase in binding capacity to hyaluronic acid. The pretreated cells are thus incorporated into hyaluronic acid.

The present disclosure relates to a method of producing a high retention cell injectate comprising treating at least one cell with at least one CD44 receptor increasing agent to produce at least one CD44 receptor enhanced cell, and incorporating the at least one CD44 receptor enhanced cell into hyaluronic acid.

In another embodiment, the at least one cell is a stem cell, cardiac myoblast, vascular smooth muscle cell, or chondrocyte. In another embodiment, the stem cell is totipotent, pluripotent, multipotent or unipotent.

In another embodiment of the present method of producing a high retention cell injectate, the at least one CD44 receptor increasing substance is lidocaine or insulin. In another embodiment, the CD44 receptor is CD44v3 receptor or CD44v6 receptor.

The present disclosure also relates to a high retention cell injectate comprising at least one cell treated with a CD44 receptor increasing agent and hyaluronic acid. In another embodiment of a high retention cell injectate, the at least one cell is a stem cell, cardiac myoblast, vascular smooth muscle cell, or chondrocyte. In another embodiment of a high retention cell injectate, the stem cell is totipotent, pluripotent, multipotent or unipotent. In another embodiment of a high retention cell injectate, the CD44 receptor increasing substance is lidocaine or insulin. In another embodiment of a high retention cell injectate, the CD44 receptor is CD44v3 receptor or CD44v6 receptor. In another embodiment of a high retention cell injectate, the cell injectate further comprises at least one bioactive agent. Alternatively, the at least one bioactive agent may be selected from the group consisting of PDGF-BB, VEGF, and IGF, or a combination thereof.

The present disclosure also relates to a method of treating a disease or condition comprising administering to a patient in need thereof at least one high retention cell injectate comprising at least one cell treated with a CD44 receptor increasing agent and hyaluronic acid. In another embodiment of a method of treating a disease or condition, the disease or condition is selected from the group consisting of heart failure, cartilage degeneration, and arterial wall thinning. In another embodiment of a method of treating a disease or condition, the at least one cell injectate is delivered by an apparatus for delivery. In another embodiment of a method of treating a disease or condition, apparatus is a catheter. In another embodiment of a method of treating a disease or condition, the apparatus is a needle. In another embodiment of a method of treating a disease or condition, the needle is a helical needle. In another embodiment of a method of treating a disease or condition, the administering is to a heart muscle of a patient.

### DETAILED DESCRIPTION OF THE INVENTION

The present disclosure generally relates to high retention cell injectates, methods of producing the high retention cell injectates and further to methods of treating diseases or conditions by administering the cell injectates to patients in need thereof.

Therefore, one embodiment of the present disclosure relates to a method of producing a high retention cell injectate comprising treating at least one cell with at least one CD44 receptor increasing agent to produce at least one CD44 receptor enhanced cell, and incorporating the at least one CD44 receptor enhanced cell into hyaluronic acid.

Hyaluronic acid (also called hyaluronan or hyaluronate) is a non-sulfated glycosaminoglycan distributed widely throughout connective, epithelial, and neural tissues. It is one of the chief components of the extracellular matrix, contributes significantly to cell proliferation and migration, and may also be involved in the progression of some malignant tumors. The average 70 kg (154 lbs) person has roughly 15 grams of hyaluronic acid in his body, one-third of which is turned over (degraded and synthesized) every day. Hyaluronic acid is derived from hyalos (Greek for vitreous) and uronic acid because it was first isolated from the vitreous humour and possesses a high uronic acid content.

The term hyaluronate refers to the conjugate base of hyaluronic acid. Until the late 1970s, hyaluronic acid was described as a ubiquitous carbohydrate polymer that is part of the extracellular matrix. For example, hyaluronic acid is a major component of the synovial fluid and was found to increase the viscosity of the fluid. Along with lubricin, it is one of the fluid's main lubricating components.

Hyaluronic acid is an important component of articular cartilage, where it is present as a coat around each cell (chondrocyte). When aggrecan monomers bind to hyaluronic acid in the presence of link protein, large highly negatively-charged aggregates form. These aggregates imbibe water and are responsible for the resilience of cartilage (its resistance to compression). The molecular weight (size) of hyaluronic acid in cartilage decreases with age, but the amount increases.

Hyaluronic acid is also a major component of skin, where it is involved in tissue repair. When skin is excessively exposed to UVB rays, it becomes inflamed (sunburn) and the cells in the dermis stop producing as much hyaluronic acid, and increase the rate of its degradation. Hyaluronic acid degradation products also accumulate in the skin after UV exposure. While it is abundant in extracellular matrices, hyaluronic acid also contributes to tissue hydrodynamics, movement and proliferation of cells, and participates in a number of cell surface receptor interactions, notably those including its primary receptor, CD44. Upregulation of CD44 itself is widely accepted as a marker of cell activation in lymphocytes. Hyaluronic acid's contribution to tumor growth may be due to its interaction with CD44. Receptor CD44 participates in cell adhesion interactions required by tumor cells.

Although hyaluronic acid binds to receptor CD44, hyaluronic acid degradation products transduce their inflammatory signal through Toll-like receptor 2 (TLR2), TLR4 or both TLR2, and TLR4 in macrophages and dendritic cells. TLR and hyaluronic acid play a role in innate immunity. High concentrations of hyaluronic acid in the brains of young rats, and reduced concentrations in the brains of adult rats suggest that hyaluronic acid plays an important role in brain development.

The chemical structure of hyaluronic acid was determined in the 1950s in the laboratory of Karl Meyer. Hyaluronic acid is a polymer of disaccharides, themselves composed of D-glucuronic acid and D-N-acetylglucosamine, linked together via alternating β-1,4 and β-1,3 glycosidic bonds. Hyaluronic acid can be 25,000 disaccharide repeats in length. Polymers of hyaluronic acid can range in size from 5,000 to 20,000,000 Da in vivo. The average molecular weight in human synovial fluid is 3-4 million Da, and hyaluronic acid purified from human umbilical cord is 3,140,000 Da.

Hyaluronic acid is energetically stable in part because of the stereochemistry of its component disaccharides. Bulky groups on each sugar molecule are in sterically favored positions, whereas the smaller hydrogens assume the less-favorable axial positions. Hyaluronic acid is synthesized by a class of integral membrane proteins called hyaluronic acid synthases, of which vertebrates have three types: HAS1, HAS2, and HAS3. These enzymes lengthen hyaluronic acid by repeatedly adding glucuronic acid and N-acetylglucosamine to the nascent polysaccharide as it is extruded through the cell membrane into the extracellular space.

Hyaluronic acid synthesis (HAS) has been shown to be inhibited by 4-Methylumbelliferone (hymecromone, heparvit), a 7-Hydroxy-4-methylcoumarin derivative. This selective inhibition (without inhibiting other Glycosaminoglycans) may prove useful in preventing metastasis of malignant tumor cells. Hyaluronic acid is degraded by a family of enzymes called hyaluronidases. In humans, there are at least seven types of hyaluronidase-like enzymes, several of which are tumor suppressors. The degradation products of hyaluronic acid, the oligosaccharides and very low-molecular-weight hyaluronic acid, exhibit pro-angiogenic properties. In addition, recent studies showed that hyaluronic acid fragments, not the native high-molecular mass of hyaluronic acid, can induce inflammatory responses in macrophages and dendritic cells in tissue injury and in skin transplant rejection.

Hyaluronic acid is naturally found in many tissues of the body, such as skin, cartilage, and the vitreous humour. Hyaluronic acid has high biocompatibility and has a common presence in the extracellular matrix of tissues.

Into a matrix of hyaluronic acid, cells such as stem cells may be incorporated. The resulting matrix may or may not be a product of cross-linked or non-cross-linked hyaluronic acid. Hyaluronic acid (hyaluronic acid, HA) solutions are known to be highly biocompatible and degrade over time.

Hyaluronic acid solutions can be used as cell carriers. One of the most important challenges in the field of cell delivery is to permit a cell injectate to remain at the location where they have been delivery. Another important aspect of this problem consists in using a biocompatible carrier for cell delivered. To allow for cell rentention into hyaluronic acid solution, the present disclosure contemplates pretreatment of cells with a substance that would increase the number of CD44 receptors on their surfaces. Then the pretreated cells are incorporated into hyaluronic acid. Lidocaine is a substance known to increase CD44 receptors on chondrocytes. Also, it has been demonstrated that cells expressing CD44 ligands significantly bind hyaluronic acid. It is therefore proposed to treat cells with lidocaine or other substances responsible for CD44 expression prior to dispersing them into HA solutions to allow for increased cell retention at injection sites.

The CD44 protein is a cell-surface glycoprotein involved in cell-cell interactions, cell adhesion and migration. It is a receptor for hyaluronic acid and can also interact with other ligands, such as osteopontin, collagens, and matrix metalloproteinases (MMPs). A specialized sialofucosylated glycoform of CD44 called HCELL is found natively on human hematopoietic stem cells, and is a highly potent E-selectin and L-selectin ligand. HCELL functions as a “bone homing receptor”, directing migration of human hematopoietic stem cells and mesenchymal stem cells to bone marrow.

This protein participates in a wide variety of cellular functions including lymphocyte activation, recirculation and homing, hematopoiesis, and tumor metastasis. Transcripts for this gene undergo complex alternative splicing that results in many functionally distinct isoforms, however, the full length nature of some of these variants has not been determined. Alternative splicing is the basis for the structural and functional diversity of this protein, and may be related to tumor metastasis. Splice variants of CD44 on colon cancer cells display the HCELL glycoform, which mediates binding to vascular E-selectin under hemodynamic flow conditions, a critical step in colon cancer metastasis. CD44 gene transcription is at least in part activated by beta catenin and Wnt signaling (also linked to tumour development). The protein is a determinant for the Indian blood group system.

CD44, along with CD25, is used to track early T cell development in the thymus. CD44 expression is an indicative marker for effector-memory T-cells. It is tracked with CFSE chemical tagging. In addition, variations in CD44 can server as cell surface markers for some breast and prostate cancer stem cells.

The number of CD44 receptors can be increased or enhanced by present treatment of the cells of interest with one or more CD44 receptor increasing agents to produce at least one CD44 receptor enhanced cell. The increased interaction between CD44 and hyaluronic acid increases the likelihood that a locally delivered cell injectate will remain in the area of delivery with hyaluronic acid and the cells of interest intact. Cells expressing CD44 ligands bind to hyaluronic acid more. Only through chemical degradation of hyaluronic acid (which is slow) can the cells of interest be released. When cells are not incorporated properly into the matrix of hyaluronic acid, the cells escape the matrix and are washed out and the slow degradation of hyaluronic acid in the body after delivery makes no difference for cell therapy. Sodium hyaluronate, a specific example of a hyaluronate, is within the scope and teaching of the present disclosure.

Lidocaine is a common local anesthetic and antiarrhythmic drug. Lidocaine is used topically to relieve itching, burning and pain from skin inflammations, injected as a dental anesthetic, and in minor surgery. Lidocaine has been demonstrated to increase the number of CD44 receptors on their surfaces. Any CD44 receptor increasing agent is within the scope and teachings of the present disclosure.

Stem cells are cells found in most multi-cellular organisms. They are capable of retaining the ability to reinvigorate themselves through mitotic cell division and can differentiate into a diverse range of specialized cell types. The two broad types of mammalian stem cells are: embryonic stem cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

As stem cells can be grown and transformed into specialized cells with characteristics consistent with cells of various tissues such as muscles or nerves through cell culture, their use in medical therapies has been proposed. In particular, embryonic cell lines, autologous embryonic stem cells generated through therapeutic cloning, and highly plastic adult stem cells from the umbilical cord blood or bone marrow are touted as promising candidates.

The classical definition of a stem cell requires that it possess two properties: self-renewal which is the ability to go through numerous cycles of cell division while maintaining the undifferentiated state, and potency which is the capacity to differentiate into specialized cell types. In the strictest sense, this requires stem cells to be either totipotent or pluripotent, i.e., to be able to give rise to any mature cell type, although multipotent or unipotent progenitor cells are sometimes referred to as stem cells.

Potency specifies the differentiation potential (the potential to differentiate into different cell types) of the stem cell. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g. hematopoietic stem cells differentiate into red blood cells, white blood cells, platelets, etc.). Unipotent cells can produce only one cell type, but have the property of self-renewal which distinguishes them from non-stem cells (e.g. muscle stem cells).

The practical definition of a stem cell is the functional definition - the ability to regenerate tissue over a lifetime. For example, the standard test for a bone marrow or hematopoietic stem cell (HSC) is the ability to transplant one cell and save an individual without HSCs. In this case, a stem cell must be able to produce new blood cells and immune cells over a long term, demonstrating potency. It should also be possible to isolate stem cells from the transplanted individual, which can themselves be transplanted into another individual without HSCs, demonstrating that the stem cell was able to self-renew.

Properties of stem cells can be illustrated in vitro, using methods such as clonogenic assays, where single cells are characterized by their ability to differentiate and self-renew. As well, stem cells can be isolated based on a distinctive set of cell surface markers. However, in vitro culture conditions can alter the behavior of cells, making it unclear whether the cells will behave in a similar manner in vivo.

To ensure self-renewal, stem cells undergo two types of cell division. Symmetric division gives rise to two identical daughter cells both endowed with stem cell properties. Asymmetric division, on the other hand, produces only one stem cell and a progenitor cell with limited self-renewal potential. Progenitors can go through several rounds of cell division before terminally differentiating into a mature cell. It is possible that the molecular distinction between symmetric and asymmetric divisions lies in differential segregation of cell membrane proteins (such as receptors) between the daughter cells.

Stem cell therapy has the potential to dramatically change the treatment of human disease. A number of adult stem cell therapies currently exist, particularly bone marrow transplants that are used to treat leukemia. The technologies derived from stem cell research may be used to treat a wider variety of diseases including cancer, Parkinson's disease, spinal cord injuries, and muscle damage, amongst a number of other impairments and conditions.

Stems cells are an example of the type cells which may be used as part of the present high rentention cell injectate. Other types of cells further include, without limitation, cardiac myoblasts, vascular smooth muscle cells, or chondrocytes.

Acute myocardial infarction (AMI or MI), more commonly known as a heart attack, is a medical condition that occurs when the blood supply to a part of the heart is interrupted, most commonly due to rupture of a vulnerable plaque. The resulting ischemia or oxygen shortage, if left untreated for a sufficient period, can cause damage and/or death of heart tissue. It is a medical emergency, and the leading cause of death for both men and women all over the world. Important risk factors are a history of vascular disease such as atherosclerotic coronary heart disease and/or angina, a previous heart attack or stroke, any previous episodes of abnormal heart rhythms or syncope, older age-especially men over 40 and women over 50, smoking, excessive alcohol consumption, the abuse of certain drugs, high triglyceride levels, high LDL (low-density lipoprotein, “bad cholesterol”) and low HDL (high density lipoprotein, “good cholesterol”), diabetes, high blood pressure, obesity, and chronic high stress levels. Chronic kidney disease and a history of heart failure are also significant risk factors which may indicate a hightend disposition towards suffering a MI.

The term myocardial infarction is derived from myocardium (the heart muscle) and infarction (tissue death due to oxygen starvation). The phrase “heart attack” is sometimes used incorrectly to describe sudden cardiac death, which may or may not be the result of acute myocardial infarction. A heart attack is different from, but can be the cause of cardiac arrest, which is the stopping of the heartbeat, and cardiac arrhythmia, an abnormal heartbeat. It is also distinct from heart failure, in which the pumping action of the heart is impaired; severe myocardial infarction may lead to heart failure, but not necessarily.

Classical symptoms of acute myocardial infarction include chest pain (typically radiating to the left arm or left side of the neck), shortness of breath, nausea, vomiting, palpitations, sweating, and anxiety (often described as a sense of impending doom). Patients frequently feel suddenly ill. Women often experience different symptoms from men. The most common symptoms of MI in women include shortness of breath, weakness, and fatigue. Approximately one fourth of all myocardial infarctions are silent, without chest pain or other symptoms. A history of diabetes should heighten the index of suspicion, particularly if the patient has diabetic neuropathy (diabetes-related nerve damage).

Immediate treatment for suspected acute myocardial infarction includes oxygen, aspirin, and sublingual glyceryl trinitrate (colloquially referred to as nitroglycerin and abbreviated as NTG). Pain relief is also often given, classically morphine sulfate. The patient will receive a number of diagnostic tests, such as an electrocardiogram (ECG, EKG), a chest X-ray and blood tests to detect elevations in the creatine kinase-MB (CK-MB) fraction or in troponin I (TnI) or troponin T (TnT) levels (these are chemical markers specific to the myocardium and are often referred to as cardiac markers). On the basis of the ECG, a distinction is made between ST elevation MI (STEMI) or non-ST elevation Ml (NSTEMI). Most cases of STEMI are treated with thrombolysis or if possible with percutaneous coronary intervention (PCI, angioplasty and stent insertion), provided the hospital has facilities for coronary angiography. NSTEMI is managed with medication, although PCI is often performed during hospital admission. In patients who have multiple blockages and who are relatively stable, or in a few extraordinary emergency cases, bypass surgery of the blocked coronary artery performed by a cardiothoracic surgeon is an option.

An apparatus for delivery is required to deliver the present high retention cell injectate including cells such as stems cells to one or more target sites such as the heart muscle wall. This apparatus can be a catheter in one embodiment. In general, a catheter is a tube that can be inserted into a body cavity, duct or vessel. Catheters thereby allow drainage or injection of fluids or access by surgical instruments. The process of inserting a catheter is catheterization. In most uses a catheter is a thin, flexible tube: a “soft” catheter; in some uses, it is a larger, solid tube: a “hard” catheter. Placement of a catheter into a particular part of the body may allow: draining urine from the urinary bladder as in urinary catheterization, e.g., the Foley catheter or even when the urethra is damaged as in suprapubic catheterization, drainage of urine from the kidney pelvis by percutaneous nephrostomy, drainage of fluid collections, e.g. an abdominal abscess, administration of intravenous fluids, medication or parenteral nutrition, angioplasty, angiography, balloon septostomy, balloon sinuplasty. Catheters can be used for among other things: direct measurement of blood pressure in an artery or vein; direct measurement of intracranial pressure; administration of anaesthetic medication into the epidural space, the subarachnoid space, or around a major nerve bundle such as the brachial plexus; and subcutaneous administration of insulin or other medications with the use of an infusion set and insulin pump.

A central venous catheter is a conduit for giving drugs or fluids into a large-bore catheter positioned either in a vein near the heart or just inside the atrium. A Swan-Ganz catheter is a special type of catheter placed into the pulmonary artery for measuring pressures in the heart.

The catheters as contemplated by the present disclosure include mapping catheters such as those that can be used in conjunction with the NOGA® XP (Cordis Inc., Johnson & Johnson Company) system. The NOGA® XP System uses a location detection technology via a location detection device (location pad) and sensors in the mapping catheter and an external location reference patch. One of ordinary skill in the art generally are familiar with obtaining information via mapping catheters. Data is presented in 3D color-coded shape reconstructions of the heart. This color-coding enables quick visualization of the activation of the mapped tissue, in correlation with its anatomical location. The intended use of the NOGA® XP System is catheter-based cardiac electromechanical mapping. The NOGA® XP System provides important information on the electrical and mechanical activities of the heart, in a procedure that uses the same techniques and tools as other cardiac catheter mapping systems. One can use the NOGA® XP System on patients who are eligible for conventional cardiac catheterization. Maps created on the NOGA® XP system utilize data produced by the manipulations and movement of the NOGASTAR® (Cordis Inc., Johnson & Johnson Company) Mapping Catheter inside the heart chamber.

With its unique electrode tip and on-board sensor, the NOGASTAR® catheter can be manipulated in six degrees—x, y, z, pitch, yaw, and roll. Once in contact with the myocardium, the sensor gathers electrical data from the heart to form the basis of an electromechanical map. Available in a variety of curves and lengths, the NOGASTAR® Mapping Catheter puts detailed electromechanical maps close at hand.

The present high retention cell injectate may also be injected with the use of a needle. The needle can be a hypodermic needle. A hypodermic needle is a hollow needle commonly used with a syringe to inject substances into the body. A hypodermic needle is used for instant delivery of a drug, or when the injected substance cannot be ingested, either because it would not be absorbed (as with insulin), or because it would harm the liver (as with testosterone). There are many possible routes for an injection.

Virtually all current hypodermic needles and their associated syringes are designed for single use because they are hard to decontaminate and need sharpening after repeat use. Needles are normally used only once and disposed of in a container. Hypodermic needles are normally made from a stainless-steel tube drawn through progressively smaller dies to make the needle. The end is bevelled to create a sharp pointed tip. This lets the needle easily penetrate the skin. When a hypodermic needle is inserted, the bevel should be facing upwards.

The diameter of the needle is indicated by the needle gauge. Various needle lengths are available for any given gauge. There are a number of systems for gauging needles, including the Stubs Needle Gauge, and the French Catheter Scale. Needles in common medical use range from 7 gauge (the largest) to 33 (the smallest) on the Stubs scale. Twenty-one-gauge needles are most commonly used for drawing blood for testing purposes, and sixteen- or seventeen-gauge needles are most commonly used for blood donation, as they are large enough to allow red blood cells to pass through the needle without rupturing (this also allows more blood to be collected in a shorter amount of time). Smaller-gauge needles will rupture the red blood cells, and if this occurs, the blood is useless for the patient that is receiving it. Although reusable needles remain useful for some scientific applications, disposable needles are far more common in medicine. Disposable needles are embedded in a plastic or aluminum hub that attaches to the syringe barrel by means of a press-fit (Luer) or twist-on (Luer-lock) fitting.

The needle can also be a helical needle such as that disclosed in WO/2006/063481. Helical needles such as those used for present delivery of cells may consist of a wire-shaped needle material arranged in a helical line in a plurality of threads with a constant pitch and diameter. Whatever the type of needle that is used, its gauge of the needle and extrusion forces needed to can be selected by one of ordinary skill in the art to ensure proper injection of the present plugs comprising collagen and cells.

### EXAMPLES

1) Hyaluronic acid (HA) carrier is prepared by mixing hyaluronate in PBS for a final viscosity of approximately 25 cP. Human arterial smooth muscle cells are grown in 96-well plates and exposed to 100 to 1000 μU/mL insulin for 24 hours. CD44 receptor rich cells are trypsinized and resuspended in the prepared HA solution. The cell-carrier mixture is then injected in the target organ or tissue.
2) HA carrier is prepared by mixing hyaluronate in PBS for a final viscosity of approximately 25 cP. Human chondrocytes are grown in 96-well plates and exposed to lidocaine. CD44 receptor-rich chondrocytes are trypsinized and resuspended in the prepared HA solution. The cell-carrier mixture is then injected in the patient cartilage.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I claim:

1. A method of producing a high retention cell injectate comprising:
a) treating at least one cell with at least one CD44 receptor increasing agent to produce at least one CD44 receptor enhanced cell, and
b) incorporating said at least one CD44 receptor enhanced cell into hyaluronic acid.

2. The method of claim 1, wherein said hyaluronic acid is cross-linked.

3. The method of claim 1, wherein said at least one cell is a stem cell, cardiac myoblast, vascular smooth muscle cell, or chondrocyte.

4. The method of claim 3, wherein said stem cell is totipotent, pluripotent, multipotent or unipotent.

5. The method of claim 1, wherein said at least one CD44 receptor increasing substance is lidocaine or insulin.

6. The method of claim 1, wherein said CD44 receptor is CD44v3 receptor or CD44v6 receptor.

7. A high retention cell injectate comprising at least one cell treated with a CD44 receptor increasing agent and hyaluronic acid.

8. The cell injectate of claim 7, wherein said hyaluronic acid is cross-linked.

9. The cell injectate of claim 7, where said at least one cell is a stem cell, cardiac myoblast, vascular smooth muscle cell, or chondrocyte.

10. The cell injectate of claim 9, wherein said stem cell is totipotent, pluripotent, multipotent or unipotent.

11. The cell injectate of claim 7, wherein said CD44 receptor increasing substance is lidocaine or insulin.

12. The cell injectate of claim 7, wherein said CD44 receptor is CD44v3 receptor or CD44v6 receptor.

13. The cell injectate of claim 6, further comprising at least one bioactive agent.

14. The cell injectate of claim 13, wherein said at least one bioactive agent is selected from the group consisting of PDGF-BB, VEGF, and IGF, or a combination thereof.

15. A method of treating a disease or condition comprising administering to a patient in need thereof at least one high retention cell injectate comprising at least one cell treated with a CD44 receptor increasing agent and hyaluronic acid.

16. The method of claim 15, wherein said hyaluronic acid is cross-linked.

17. The method of claim 15, wherein said disease or condition is selected from the group consisting of heart failure, cartilage degeneration, and arterial wall thinning.

18. The method of claim 15, wherein said at least one cell injectate is delivered by an apparatus for delivery.

19. The method of claim 18, wherein said apparatus is a catheter.

20. The method of claim 18, wherein said apparatus is a needle.

21. The method of claim 20, wherein said needle is a helical needle.

22. The method of claim 15, wherein said administering is to a heart muscle of said patient.
